(19) United States
(12) Patent Application Publication
(10) Pub. No.: US 2020/0081015 A1
(43) Pub. Date: Mar. 12, 2020

(54) CANCER SCREENING PROCESSOR, CANCER SCREENING SYSTEM, AND CANCER SCREENING PROCESSING METHOD

(71) Applicant: HITACHI, LTD., Tokyo (JP)

(72) Inventors: Minoru SAKAIRI, Tokyo (JP); Mayumi ABE, Tokyo (JP); Taku NAKAMURA, Tokyo (JP)

(21) Appl. No.: 16/551,780

(22) Filed: Aug. 27, 2019

(30) Foreign Application Priority Data
Sep. 11, 2018 (JP) 2018-169802

(51) Int. Cl. G01N 33/68; G01N 30/72; G01N 33/493

(52) U.S. Cl. CPC G01N 33/6848; G01N 2800/7028; G01N 33/493; G01N 30/72

(57) ABSTRACT

A cancer screening processor includes a database which stores a sample information database storing information on a number of dispensed samples of urine sent from subjects; the sample dispensed for a cancer type which the subject desire a screening, a center selection unit which selects an analysis center C2 for each of cancer type to be analyzed by using a urinary tumor marker; the analysis center C2 receiving a sample container including dispensed sample on the basis of the cancer type and information from analysis centers, and a processing unit which counts a number of the sample containers on the basis of the sample information database, and performs a process step of sending the sample container to the analysis center when the number of the sample containers becomes equal to a number in the information from the analysis center.

### CROSS-REFERENCE TO RELATED APPLICATIONS

This application is based upon and claims the benefit of priority from Japanese Patent Application No. 2018-169802 filed on Sep. 11, 2018; the entire contents of which are incorporated herein by reference.

### BACKGROUND OF THE INVENTION

### 1. Field of the Invention

The present invention relates to techniques of a cancer screening processor, a cancer screening system, and a cancer screening method which are used for a cancer screening using a urinary tumor marker.

### 2. Description of the Related Art

WO 2017/213246 A1 discloses a disease diagnosis method by using a urinary metabolite for “detecting a cancer, predicting a cancer risk, determining a cancer stage, determining a cancer prognosis, and/or evaluating the effectiveness of a treatment in a subject by measuring a urinary metabolite in the subject”.

### SUMMARY OF THE INVENTION

In the cancer screening using a urinary metabolite, a plurality of urinary tumor markers (hereinafter appropriately referred to as markers) are quantified, and the risk of cancer is determined by using a prediction value obtained according to Prediction Formula (1) shown below. In addition, although three markers are used here, two or less markers or four or more markers are similarly considered.

Prediction value=α×(quantitative value of urinary tumor marker 1)+β×(quantitative value of urinary tumor marker 2)+γ×(quantitative value of urinary tumor marker 3)+δ (1)

In Prediction Formula (1), α, β, γ, and δ are each a predetermined constant.

FIG. 23 is a graph illustrating a method of risk assessment for cancer using Prediction Formula (1).

In this screening, as illustrated in FIG. 23, when a prediction value is larger than “0” (region W1), it is determined that the risk of cancer is high (risk “high”). Otherwise, that is, when the prediction value is “0” or less (region W2), it is determined that the risk is low (risk “low”). Incidentally, the horizontal axis in FIG. 23 is shown for the purpose of clearly showing the areas and has no particular meaning.

Such risk assessment is performed by comparing a prediction value which is an analysis result obtained as a result of measurement with a threshold value determined on the basis of past statistical data (data group of prediction values of healthy people and cancer patients). Note that, in FIG. 23, risk assessment of two stages of a risk “high” and a risk “low” is performed. In general, a definitive diagnosis of a cancer stage is made by pathological diagnosis, but unlike an advanced cancer, such as “stage 3” or “stage 4”, the diagnosis of early stage cancer, such as “stage 0” or “stage 1”, may differ by pathologists. Therefore, such risk assessment may have three stages (risks “high”, “medium”, and “low”) by adding a risk “medium” to the two-stages risk assessment.

As described above, when a plurality of markers are used, an analyzer called liquid chromatograph mass spectrometer (LC/MS) is used to quantify the markers. There, firstly, a number of metabolites in a solution are separated into components by a liquid chromatograph (LC). Then the separated components are then sequentially detected by a mass spectrometer.

In the analysis using the liquid chromatograph mass spectrometer described above, it is necessary for an operator to adjust various conditions of the liquid chromatograph mass spectrometer to analyze a specific marker efficiently. The conditions adjusted here are separation conditions (solvent types used at a mobile phase, separation column, etc.) in liquid chromatograph and measurement conditions (sensitivity of a detector, measurement mass range, etc.) in the mass spectrometer.

These adjustments require time and labor. Therefore, in analysis by the liquid chromatograph mass spectrometer, it is desirable to continuously analyze the same markers under the same conditions as much as possible to increase an actual operation time (time during which the sample is measured) of the liquid chromatograph mass spectrometer. This is ultimately effective for the cost of the analysis. This situation is illustrated in FIG. 24.

FIG. 24 illustrates an example of analyzing lots L for different cancer types.

Here, a lot processing L1 represents the lot processing of “cancer type A”, a lot processing L2 the lot processing of “cancer type B”, and a lot processing L3 the lot processing of “cancer type C”. The lot processing means continuous analysis of a plurality of urine samples contained in a lot by the liquid chromatograph mass spectrometer.

Before the lot processing L1 to L3, an adjustment P of the above-mentioned separation conditions and analysis conditions is performed. Here, an adjustment P1 is the adjustment P for “cancer type A”. Furthermore, an adjustment P2 is the adjustment P for “cancer type B”, and an adjustment P3 is the adjustment P for “cancer type C”.

In the example of FIG. 24, the adjustments P1 to P3 for the respective cancer types are performed for each of the different lot processing L1 to L3. As described above, since these adjustments P1 to P3 require time and labor, it is not efficient to perform the adjustments P1 to P3 every time to perform the respective lot processing L1 to L3.

Note that, in FIG. 24, condition check N is performed after the end of the lot processing L1 to L3. The condition check N is mainly sensitivity check for the liquid chromatograph mass spectrometer. The condition check N is performed at regular intervals, and the contents thereof is not related to the cancer types. However, while the condition check N is being performed, analysis by the liquid chromatograph mass spectrometer cannot be performed. Therefore, as illustrated in FIG. 24, even if it is desired to perform a further analysis, after completion of a lot processing, another lot processing, the other lot processing cannot be performed when an adjustment P and a lot processing overlap with a condition check N in time.

Therefore, as illustrated in FIG. 24, time is wasted between the lot processing and the condition check N.

When lot processing is performed according to a schedule as illustrated in FIG. 24, the operation rate of the liquid chromatograph mass spectrometer stays at about 30 percent per week.

Unless the problem as illustrated in FIG. 24 is overcome, it is not realistic to carry out a large amount of marker analysis by using the liquid chromatograph mass spectrometer in such a manner.

In addition, when urine samples are directly sent from subjects to an analysis center where analysis is actually performed, a person in charge in the analysis center will be busy sorting the respective urine samples into different cancer types. For this reason, the person in charge may spend less time performing the analysis. This results in a very expensive analysis. Furthermore, the analysis is performed well with a small number of urine samples, but when the number of urine samples is considerably large, for example, the urine samples are increased in number from 100,000 samples/year to 1,000,000 samples/year, the analysis is insufficiently performed by using the process as illustrated in FIG. 24.

The present invention is made in view of such a background, and an object of the present invention is carrying out an efficient cancer screening with tumor markers in urine samples.

In order to solve the problems described above, the present invention includes a storage unit storing sample information including information on a number of urine samples sent from subjects, a center selection unit selecting an analysis center for each of the cancer types on which cancer screening is performed on the basis of the cancer type and analysis center information transmitted from analysis centers; receiving a sample container containing the urine sample being dispensed for each of the cancer types, analyzing a urinary tumor marker in the dispensed urine sample for the cancer types, and a processing unit counting a number of the sample containers and performing a process step of sending the sample containers to the analysis center when the number of the sample becomes equal to a number in the analysis center information transmitted from the analysis center.

Other solutions will be described as appropriate in the embodiments.

According to the present invention, an efficient cancer screening using a urinary tumor marker can be carried out.

### BRIEF DESCRIPTION OF THE DRAWINGS

FIG. 1 is a diagram illustrating a configuration example of a cancer screening system according to the present embodiment;

FIG. 2 is a functional block diagram illustrating a configuration example of a comprehensive analysis center server;

FIG. 3A is a timing chart (part 1) illustrating operation of the cancer screening system according to the present embodiment;

FIG. 3B is a timing chart (part 2) illustrating operation of the cancer screening system according to the present embodiment;

FIG. 4 is a flowchart illustrating a detailed operation procedure of storage and management in the comprehensive analysis center server;

FIG. 5 is a table illustrating an example of a membership information database;

FIG. 6 is a table illustrating an example of a sample information database;

FIG. 7A is a table (part 1) illustrating an example of a sample information database stored in an analysis center server;

FIG. 7B is a table (part 2) illustrating an example of a sample information database stored in the analysis center server;

FIG. 8 is a table illustrating an example of a urine sample collection container information database;

FIG. 9 is a table illustrating an example of an analysis center information database;

FIG. 10 is a table illustrating an example of a price information database;

FIG. 11 is a table illustrating an example of an analysis condition database;

FIG. 12 is a table illustrating an example of analysis result information;

FIG. 13 is a diagram illustrating an example of an application screen;

FIG. 14 is a diagram illustrating an example of an application program screen;

FIG. 15 is a diagram illustrating an example of an acceptance check screen;

FIG. 16 is a diagram illustrating an example of an analysis request inquiry screen;

FIG. 17 is a diagram illustrating an example of a screening result display screen;

FIG. 18 illustrates an example of testing lots for the same cancer type;

FIG. 19 is a diagram illustrating a screening method for different cancer types;

FIG. 20 is a graph illustrating a result of risk assessment for breast cancer performed by a method as illustrated in FIG. 19;

FIG. 21 is a graph illustrating a result of risk assessment for colorectal cancer performed by the method as illustrated in FIG. 19;

FIG. 22 is a diagram illustrating a configuration example of a cancer screening system according to a second embodiment;

FIG. 23 is a graph illustrating a cancer risk assessment method using a prediction formula; and

FIG. 24 illustrates an example of analyzing lots for different cancer types.

### DETAILED DESCRIPTION OF THE EMBODIMENTS

Next, modes for carrying out the present invention (hereinafter referred to as “embodiments”) will be described in detail with appropriate reference to the drawings.

### First Embodiment

[Cancer Screening System Z]

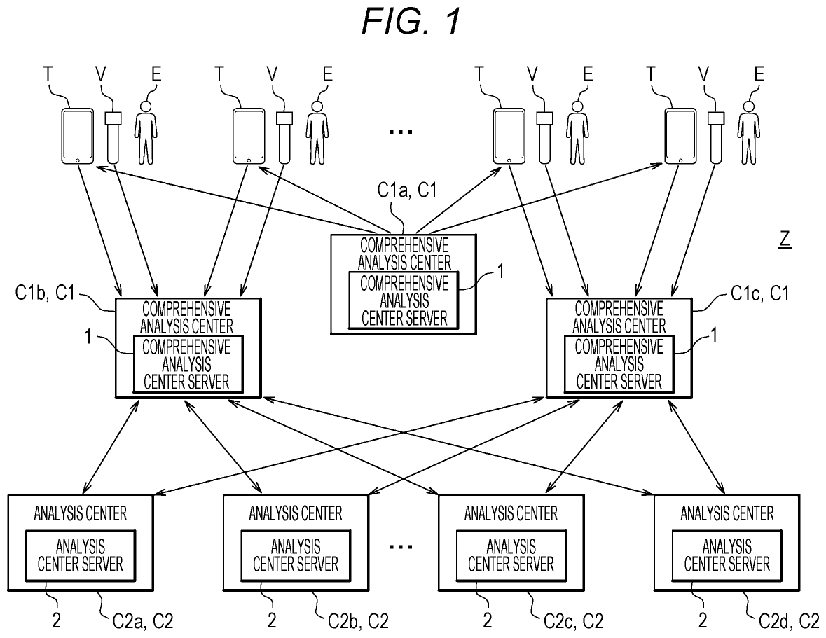

FIG. 1 is a diagram illustrating a configuration example of a cancer screening system Z according to the present embodiment.

The cancer screening system Z is composed of a three layers of configurations which are a plurality of (three in the example of FIG. 1) comprehensive analysis centers C1 (C1a to C1c), a plurality of analysis centers C2 (C2a to C2d), and mobile terminals (user devices) T of a plurality of subjects E.

(Comprehensive Analysis Center C1)

For example, each of the comprehensive analysis center C1 receives applications for the screening from subjects E and collects urine sample collection containers V each containing a urine sample (hereinafter referred to as a sample). The urine sample collection containers V (samples) are collected by mail. The samples are individually managed on the basis of sample numbers stored in barcodes or the like. Then, in the comprehensive analysis center C1, each of the samples received is dispensed for cancer types for which a corresponding subject E desires toots analyses. The dispensed samples are stored and frozen or cryopreserved in sample containers for the respective cancer types. Then, the comprehensive analysis center C1 collectively sends a predetermined amount of samples (for example, 50 to 10,000 samples) collected for each cancer type, to an analysis center C2 having time to analyze the samples.

The other operations and processing performed by the comprehensive analysis center C1 will be described later.

In FIG. 1, there are three comprehensive analysis centers C1. The three comprehensive analysis centers C1 are divided, for example, as follows. The comprehensive analysis center C1b collects samples in eastern Japan and sends the samples to an analysis center C2. Likewise, the comprehensive analysis center C1c collects samples in western Japan and sends the samples to an analysis center C2. The comprehensive analysis center C1a presides the comprehensive analysis centers C1b and C1c. The comprehensive analysis center C1a performs risk assessment on the basis of prediction values transmitted from the analysis centers C2. The prediction value is a value obtained from Prediction Formula (1) described above. An analysis result is transmitted to a mobile terminal T of each of the subjects E with a result of the risk assessment as an analysis result.

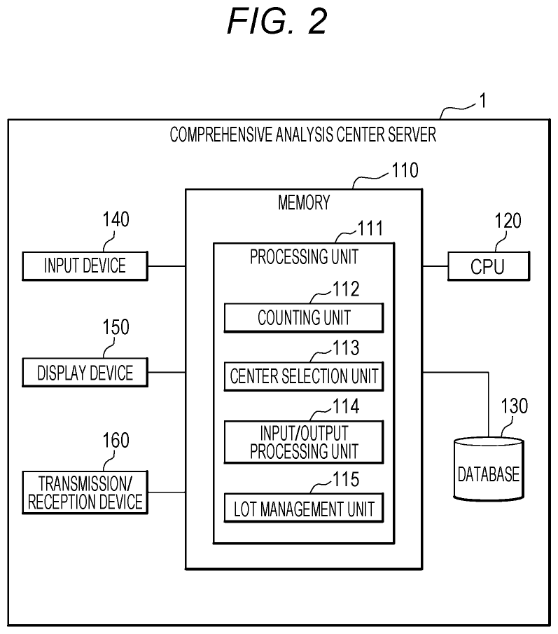

However, FIG. 1 illustrates an example, and the comprehensive analysis centers C1 do not need to be assigned roles as illustrated in FIG. 2. One comprehensive analysis center C1 may collect the samples, send the samples to the analysis centers C2, and transmit the analysis results. Alternatively, one comprehensive analysis center C1 may collect the samples and send the samples to the analysis centers C2, and another comprehensive analysis center C1 may receive the analysis results and transmit the analysis results.

Each of the comprehensive analysis centers C1a to C1c includes a comprehensive analysis center server (cancer screening processor) 1 performing processing in each of the comprehensive analysis centers C1a to C1c. The comprehensive analysis center server 1 will be described later.

(Analysis center C2)

In each of the analysis centers C2, a preparation for liquid chromatograph mass spectrometers (not illustrated) is done in advance according to a cancer type tested for. Such a configuration enables a preparation for analysis including pretreatment when the samples arrive. When the pretreatment is finished, a plurality of the samples having been received is simultaneously quantified by the liquid chromatograph mass spectrometer.

Results of the analysis are transmitted to the comprehensive analysis centers C1.

Note that each analysis center C2 makes an analysis for a specific cancer type, for example, the analysis center C2a makes an analysis for breast cancer and colorectal cancer, the analysis center C2b stomach cancer and pancreatic cancer, and the analysis center C2c colorectal cancer.

Each analysis center C2 includes an analysis center server 2. The analysis center server 2 transmits an analysis result and the like from the liquid chromatograph mass spectrometer to the comprehensive analysis center servers 1. Other processing performed by the analysis center server 2 will be described later.

(Mobile Terminal T)

The mobile terminal T is, for example, a smartphone or tablet PC, and a dedicated application program is downloaded and installed in advance. Each subject E, for example, applies for a cancer screening, a photo image of the urine sample collection container V and transmits the image thereof, and checks an analysis result, through this application program.

In addition, in the present embodiment, as the mobile terminal T, the smartphone or tablet PC is assumed, but an individual PC or the like may be used.

[Comprehensive Analysis Center Server 1]

FIG. 2 is a functional block diagram illustrating a configuration example of the comprehensive analysis center server 1.

The comprehensive analysis center server 1 is, for example, a PC. The comprehensive analysis center server 1 includes a memory 110, a CPU 120, a database (storage unit) 130, an input device 140, such as a keyboard or a mouse, a display device 150, such as a display, and a transmission/reception device 160, such as an NIC.

A program is loaded into the memory 110. The loaded program is executed by the CPU 120, achieving a processing unit 111, a counting unit 112 constituting the processing unit 111, a center selection unit 113, an input/output processing unit (processing unit, display processing unit) 114, and a lot management unit (processing unit) 115.

Furthermore, the database 130 stores various databases 131 to 134 and the like, which is described later.

<Timing Chart>

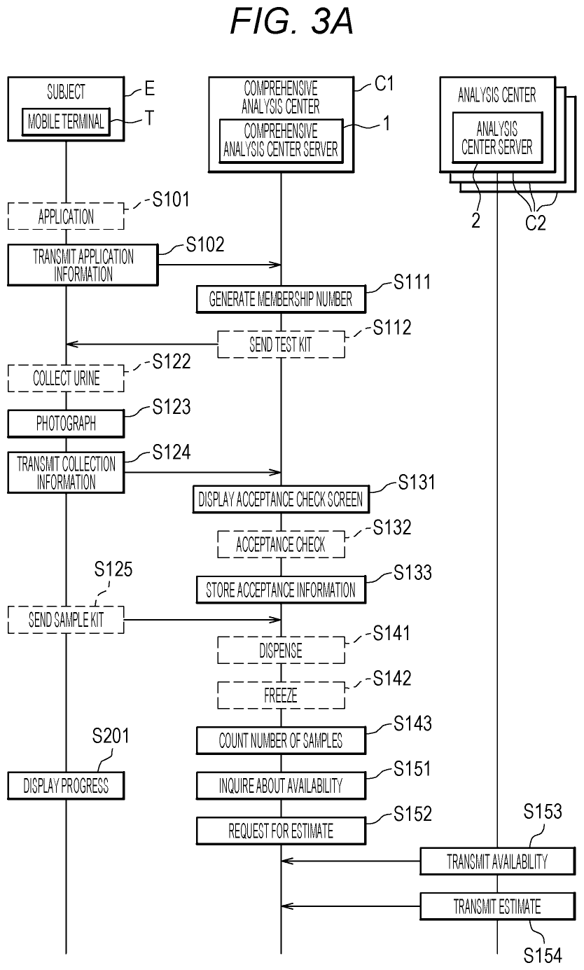

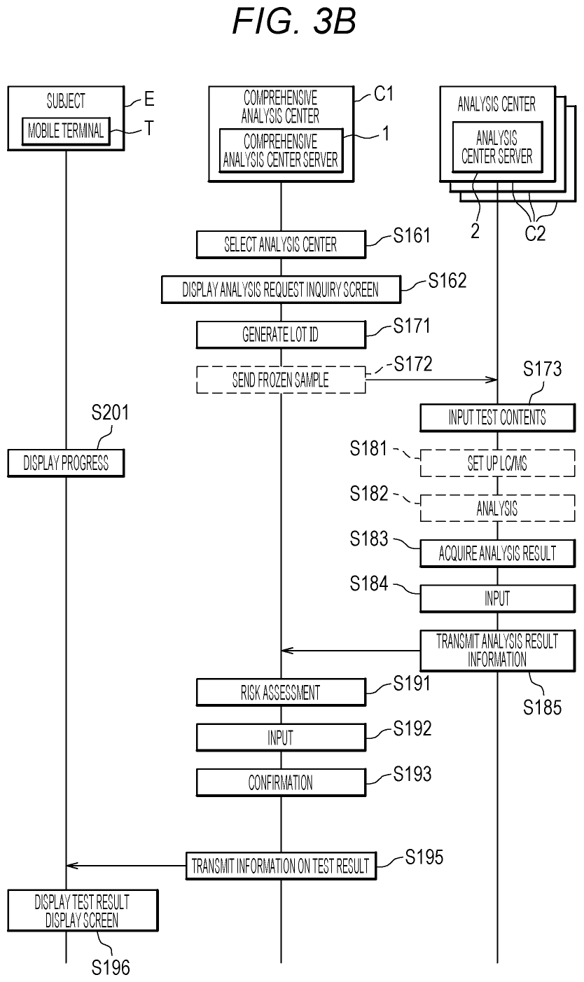

FIG. 3A and FIG. 3B are timing charts each illustrating the operation of the cancer screening system Z according to the present embodiment. FIGS. 1 and 2 are appropriately referred to.

In FIGS. 3A and 3B, the processing represented by a solid line shows processing performed by a computer, such as a mobile terminal T, a comprehensive analysis center server 1, or an analysis center server 2. Processing represented by a broken line shows processing performed by a human or a machine other than the computer.

Although FIG. 1 shows the example using three comprehensive analysis centers Cl, hereinafter, one comprehensive analysis center C1 is employed.

Firstly, a subject E applies for a cancer screening by using a mobile terminal T, such as a smartphone (S101 in FIG. 3A), and the mobile terminal T transmits information on the contents of the application as application information to a comprehensive analysis center server 1 (S102).

(Application Screen)

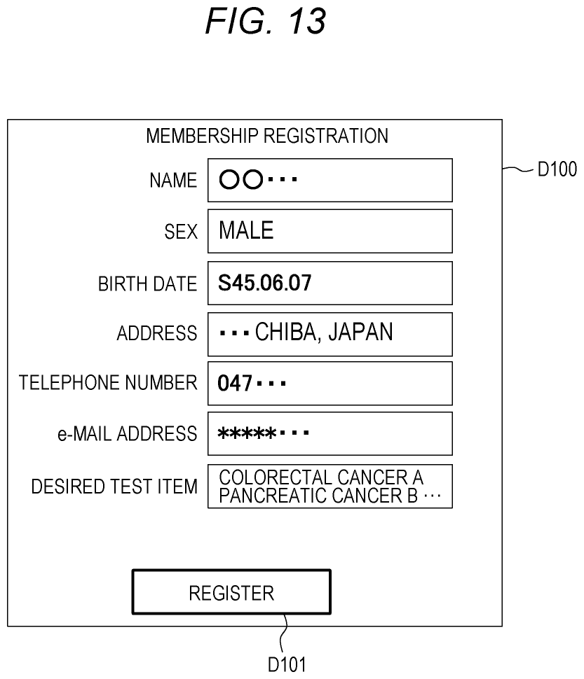

FIG. 13 is a diagram illustrating an example of an application screen D100.

The application screen D100 is a screen displayed in step S101 of FIG. 3A.

The application screen D100 is displayed on a display device, which is not illustrated, of the mobile terminal T.

As illustrated in FIG. 13, the subject E inputs information, such as his/her “name”, “sex”, “birth date”, “address”, “telephone number”, “e-mail address”, and “desired test item”, to input items in the application screen D100. Then, when the subject E selects a register button D101 displayed in the application screen D100 to make an input, the contents of the application input into the application screen D100 are transmitted as application information to the comprehensive analysis center server 1 (S102 in FIG. 3A).

Incidentally, a membership number is issued in advance upon membership registration.

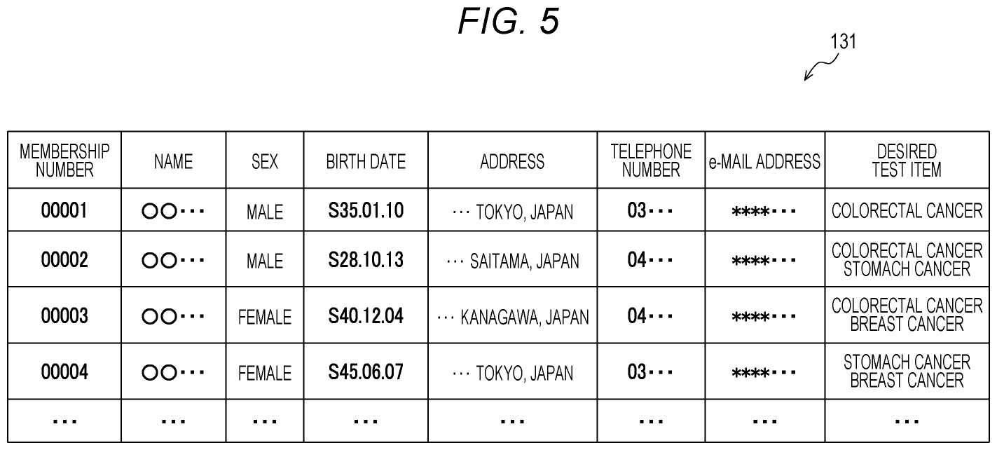

The description returns to FIG. 3A. The comprehensive analysis center server 1 receiving the application information generates the membership number for the received application information (S111) and associates the membership number with the received application information to be input into a membership information database 131 illustrated in FIG. 5.

(Membership Information Database 131)

FIG. 5 is a table illustrating an example of the membership information database 131.

The membership information database 131 is stored in the database 130 of the comprehensive analysis center server 1.

As illustrated in FIG. 5, the membership information database 131 stores the application information, such as “name”, “sex”, “birth date”, “address”, “telephone number”, “e-mail address” and “desired test item”, transmitted from the mobile terminal T in association with the membership number.

The description returns to FIG. 3A.

The comprehensive analysis center C1 sends a test kit including a urine sample collection container V to the subject E by mail (S112).

The subject E receiving the sample kit collects urine (S122) and photographs the urine sample collection container V containing the collected urine by using an application program activated on the mobile terminal T (S123). Note that, as described above, it is assumed that the application program has been downloaded and installed by the subject E in advance. Incidentally, the processing of step S123 may be omitted.

Thereafter, the subject E transmits collection information, such as the membership number, image data, collection time, and a collection location, to the comprehensive analysis center server 1 (S124). In addition to the transmission of the collection information, the subject E sends the sample kit including the urine sample collection container V containing the collected urine to the comprehensive analysis center C1 by mail (S125).

(Application Program Screen D200)

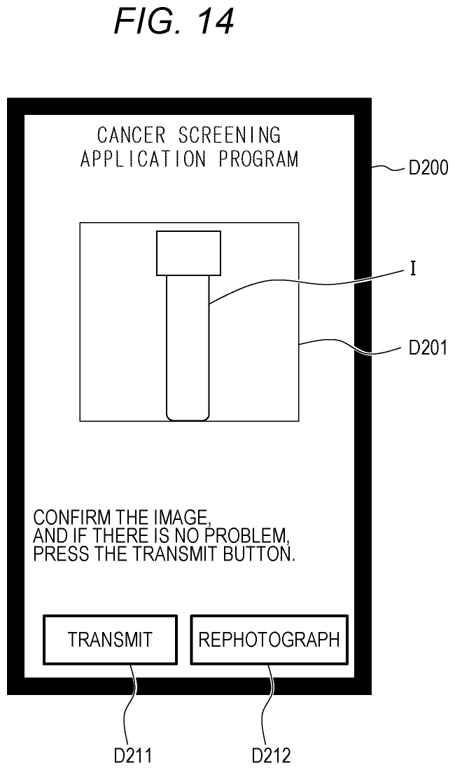

FIG. 14 is a diagram illustrating an example of an application program screen D200.

The application program screen D200 is displayed on the display device (not illustrated) of the mobile terminal T on which the application has been activated in step S123 of FIG. 3A.

The photographed image (image I of the urine sample collection container V) is displayed in an image display area D201 of the application program screen D200.

Then, when the photographed image is in a bad condition, the subject E selects a displayed re-photograph button D212 to make an input to re-photograph the urine sample collection container V.

When the photographed image is in a good state, or OK, the subject E selects a displayed transmit button D211 to make an input. Thus, the collection information is transmitted to the comprehensive analysis center server 1 (S124 in FIG. 3A). Note that, the collection time of the collection information represents, for example, the time at which the transmit button D211 is selected to make an input, acquired by a clock function of the mobile terminal T. In addition, the collection location represents information or the like of the current position acquired by a GPS function of the mobile terminal T.

The description returns to FIG. 3A.

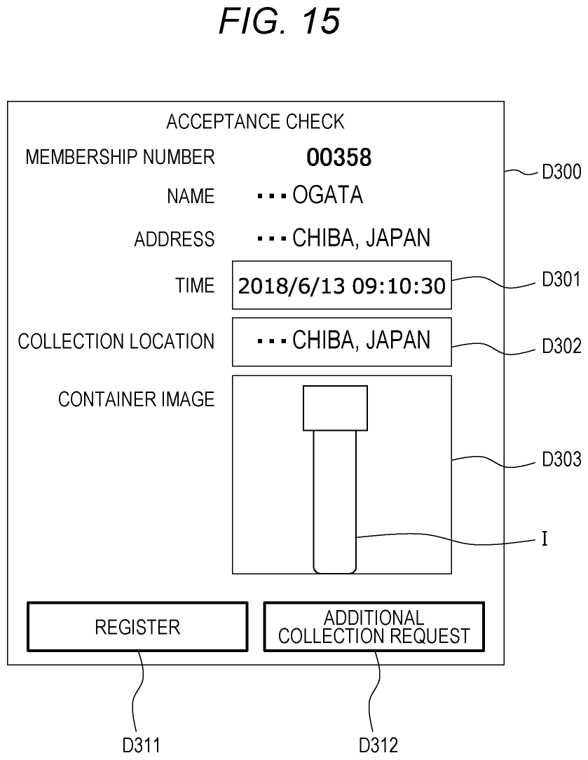

The comprehensive analysis center server 1 receiving the collection information displays an acceptance check screen D300 (see FIG. 15) on the display device 150 of the comprehensive analysis center server 1 (S131). The processing of step S131 is processing performed by an input/output processing unit 114 of the comprehensive analysis center server 1.

A checker of the comprehensive analysis center C1 refers to the acceptance check screen D300 to perform acceptance check to determine whether to accept the sample (S132).

(Acceptance Check Screen D300)

FIG. 15 is a diagram illustrating an example of the acceptance check screen D300.

As illustrated in FIG. 15, the acceptance check screen D300 displays “membership number”, “name”, and “address” thereon. Furthermore, the acceptance check screen D300 includes a time display window D301, a collection location display window D302, and a container image display area D303.

The time display window D301 displays collection time. The checker checks whether the time display in the window D301 is feasible. For example, it is checked whether different samples have the same collection time.

The collection location display window D302 displays an address of a collection location. The checker checks whether the address displayed in the window D302 is feasible. For example, it is checked whether different samples have the same collection location.

The container image display area D303 displays an image I of a transmitted urine sample collection container V. The checker checks the image I in the container image display area D303. For example, it is checked whether the urine sample collection container V has a crack or whether a sufficient amount of the sample is collected. Such a check enables elimination of a sample unsuitable for an analysis of a cancer screening, and it is possible to prevent a useless test from being carried out.

When any of the collection time, collection location, and image I of the urine sample collection container V is not in good condition, as a result of the acceptance check, the checker selects an additional collection request button D312 to make an input. Therefore, the test kit is sent to the subject E again.

When all of the collection time, collection location, and image I of the urine sample collection container V are in a good condition or OK as a result of the acceptance check, the checker selects a register button D311 to make an input. Thus, a sample number is generated for the sample.

Then, the comprehensive analysis center server 1 inputs acceptance information of the membership number, sample number, an application date, sample collection date, the collection location, and the image I of the urine sample collection container V into a sample information database (sample information) 132 and a sample container information database 133 (S133 in FIG. 3A).

(Sample Information Database 132)

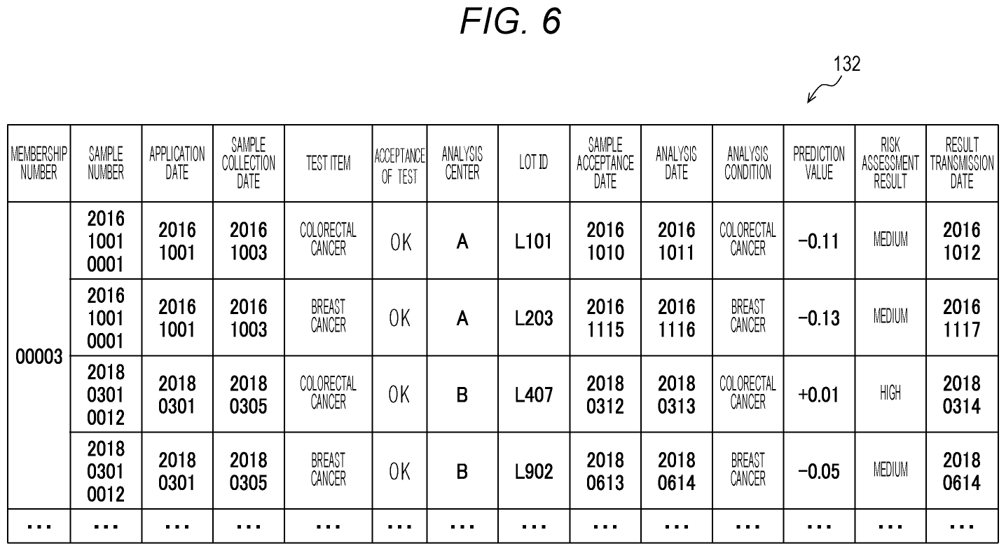

FIG. 6 is a table illustrating an example of the sample information database 132.

The sample information database 132 is stored in the database 130 of the comprehensive analysis center server 1.

As illustrated in FIG. 6, the sample information database 132 has the fields of “membership number”, “sample number”, “application date”, “sample collection date”, “test item”, “acceptance of test”, “analysis center”, “lot ID, “sample acceptance date, “analysis date”, “analysis condition”, “prediction value”, “risk assessment result”, and “result transmission date”.

In the example of FIG. 6, one subject E examines “colorectal cancer” and “breast cancer” twice each.

Here, the sample information database 132 is information shared between the comprehensive analysis center server 1 and the analysis center servers 2. Therefore, when information in the sample information database 132 is updated, updated contents are synchronously shared between the comprehensive analysis center server 1 and each of the analysis center servers 2.

In step S133, information ranging from “membership number” to “acceptance of test” is input.

The information ranging from “membership number” to “sample collection date” is information input on the basis of the collection information transmitted in step S133 of FIG. 3A.

“Test item” represents information input referring to “desired test item” of the membership information database 131 with the membership number as a key.

For “acceptance of test”, the result of the acceptance check performed in step S132 of FIG. 3A is input. In principle, the acceptance check result is not good, an additional request is made. Therefore, “OK” is input for “acceptance of test”, in principle.

The column ranging from “analysis center” to “result transmission date” are blank in step S133.

Note that “risk assessment result” stores a result of risk assessment using a prediction value. Incidentally, the risk assessment results of three stages of risks “high”, “medium”, and “low” are stored here.

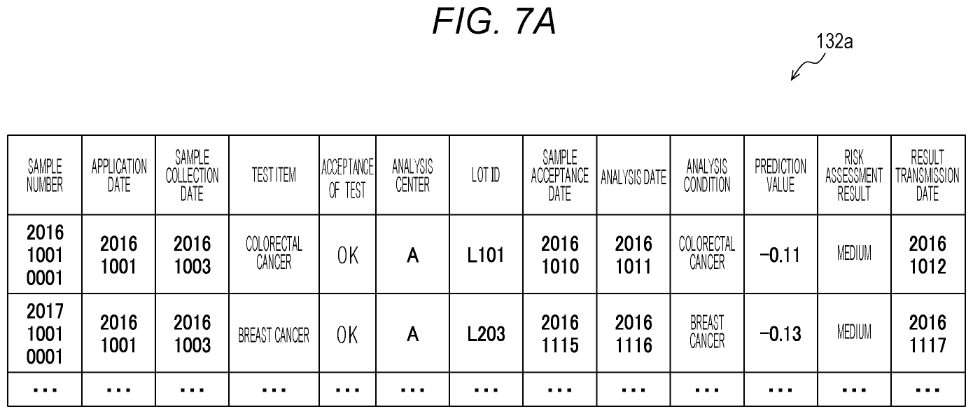

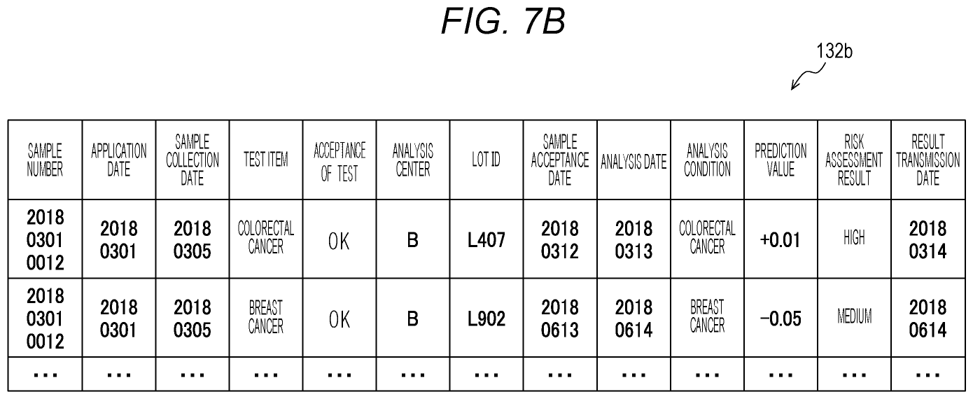

For example, FIG. 7A is a diagram illustrating an example of a sample information database 132a stored in the analysis center server 2 installed in the analysis center C2a (“analysis center A”). For example, FIG. 7B is a diagram illustrating an example of a sample information database 132b stored in the analysis center server 2 installed in the analysis center C2b (“analysis center B”).

The sample information database 132a of FIG. 7A stores only records of the sample information whose column of “analysis center” of the sample information database 132 illustrated in FIG. 6 indicates “A” which means the analysis center c2a.

Likewise, the sample information database 132b of FIG. 7B stores only records of the sample information database 132 illustrated in FIG. 6, in which “B” indicating the analysis center C2b is stored in the field of “analysis center”.

The respective column of the sample information database 132a illustrated in FIG. 7A and the sample information database 132b illustrated in FIG. 7B are similar to those in FIG. 6 except that “membership number” is omitted.

(Sample Container Information Database 133)

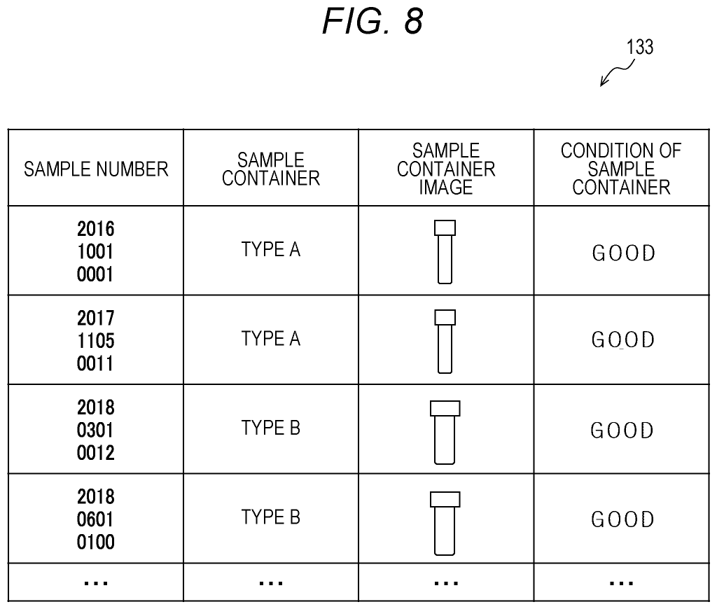

FIG. 8 is a table illustrating an example of the sample container information database 133.

The sample container information database 133 is stored in the database 130 of the comprehensive analysis center server 1.

The sample container information database 133 has the column of “sample number”, “sample container”, “sample container image”, and “condition of the sample container”.

“Sample number”, “sample container image”, and “condition of the sample container” represent information input by the comprehensive analysis center server 1 in step S133. “Sample container” may be manually input by the checker or may be input by the comprehensive analysis center server 1 by determining the urine sample collection container on the basis of the image thereof.

In addition, “sample container image” may be in blank when the sample container V is not imaged in step S123.

The description returns to FIG. 3A.

Next, the sample in the sent urine sample collection container V is dispensed for each cancer type for which a test is to be carried out (S141). The sample is dispensed into sample containers for the respective cancer types. Note that the sample containers are prepared for respective subjects E, and the samples of different subjects E are prevented from being mixed in one sample container. This dispensing may be performed manually by a staff or may be performed by a dispensing device not illustrated. Here, the cancer type tested for is a cancer type for which the subject E desires a test. For example, if a subject E desires tests for “colorectal cancer” and “breast cancer”, the sample provided by this subject E is dispensed into sample containers for “colorectal cancer” and “breast cancer”.

Then, the dispensed samples (sample containers) are stored and frozen (S142).

In addition, the comprehensive analysis center server 1 counts the number of samples (the number of sample containers) for each cancer type (S143).

Then, the comprehensive analysis center server 1 inquires of each analysis center C2 about the availability thereof (S151).

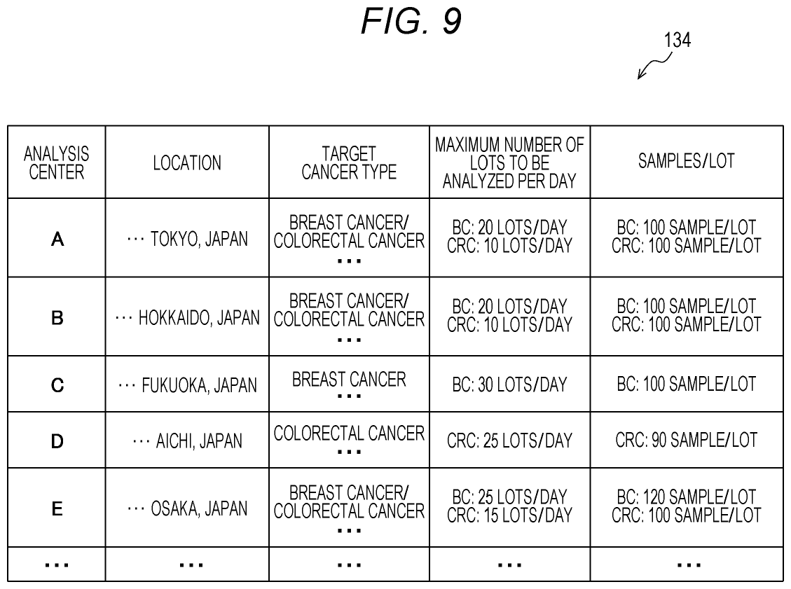

Furthermore, the comprehensive analysis center server 1 requests an estimate from each analysis center C2 (S152). Upon requesting the estimate, the number of samples for each cancer type is transmitted to each analysis center server 2. At this time, the number of samples transmitted is the number of samples analyzed at one time in each analysis center C2 according to an analysis center information database 134 of FIG. 9.

(Analysis Center Information Database 134)

FIG. 9 is a table illustrating an example of the analysis center information database 134.

The analysis center information database 134 is stored in the database 130 of the comprehensive analysis center server 1.

As illustrated in FIG. 9, the analysis center information database 134 has the columns of “analysis center”, “location”, “target cancer type”, “maximum number of lots to be analyzed per day”, and “samples/lot”.

“Location” represents the location of a corresponding analysis center C2.

“Target cancer type” represents a cancer type for which an analysis is made by the corresponding analysis center C2. In other words, the analyzable cancer type means that a urinary tumor marker of the cancer type can be analyzed.

Then, in “maximum number of lots to be analyzed per day”, the number of lots which can be analyzed per day for each cancer type by a corresponding analysis center C2 is input.

“Samples/lot” represents the number of samples per lot. Therefore, the number of samples analyzed in one day by the analysis center C2 is “samples/lot” x “maximum number of lots to be analyzed per day”.

“Samples/lot” is set in advance so that a time interval between the condition check N described above and the analysis of the lots is reduced as much as possible, on the basis of hours between the condition checks N, and an analysis time required for one sample.

For example, breast cancer and colorectal cancer can be analyzed in “analysis center A (analysis center c2a), enabling analysis of 2,000 samples per day for breast cancer (BC), and 1,000 samples for colorectal cancer (“CRC”).

The description returns to FIG. 3A.

When receiving the inquiry about the availability and the request for an estimate, each of the analysis center servers 2 refers to operation schedule information to transmit the availability of itself to the comprehensive analysis center server 1 (S153).

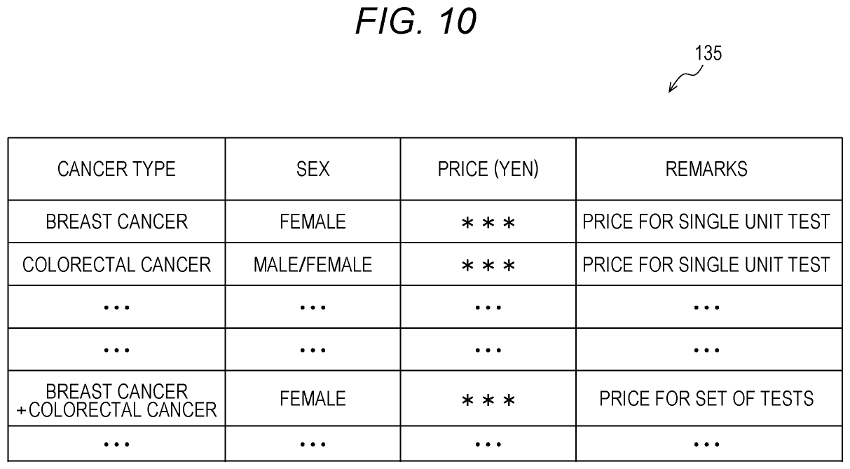

In addition, each analysis center server 2 calculates the estimate taking into consideration of conditions, such as a busy season and a slow season, and transmits the estimate to the comprehensive analysis center server 1 (S154). At this time, each analysis center server 2 calculates the estimate referring to a price information database 135 illustrated in FIG. 10.

Note that the processing in steps S151 to S154 is desirably performed every predetermined period, such as every month. Such information is used to select an analysis center C2 by the comprehensive analysis center server 1.

(Price Information Database 135)

FIG. 10 is a table illustrating an example of the price information database 135.

The price information database 135 is stored in each analysis center server 2.

As illustrated in FIG. 10, the price information database 135 has the fields of “cancer type”, “sex”, “price”, and “remarks”.

“Remarks” stores information representing, for example, whether a price stored in the field of “price” is for a single unit test or for a set of tests. Alternatively, in the field of “remarks”, price information about the busy season and the slow season, for example, the price increases 20 percent during a busy season and the price decreases 20 percent during the slow season may be described.

The description returns to FIG. 3B. FIG. 3B continues from FIG. 3A.

When the number of lots (the number of samples) reaches a predetermined number or more in a cancer type, the comprehensive analysis center server 1 selects an analysis center C2 (S161 in FIG. 3B). At this time, the comprehensive analysis center server 1 selects the analysis center C2 on the basis of the availability transmitted in step S153, the estimate transmitted in step S154, and the like. This processing will be described later.

Then, the comprehensive analysis center server 1 displays an analysis request inquiry screen D400 on the display device 150 (S162).

(Analysis Request Inquiry Screen D400)

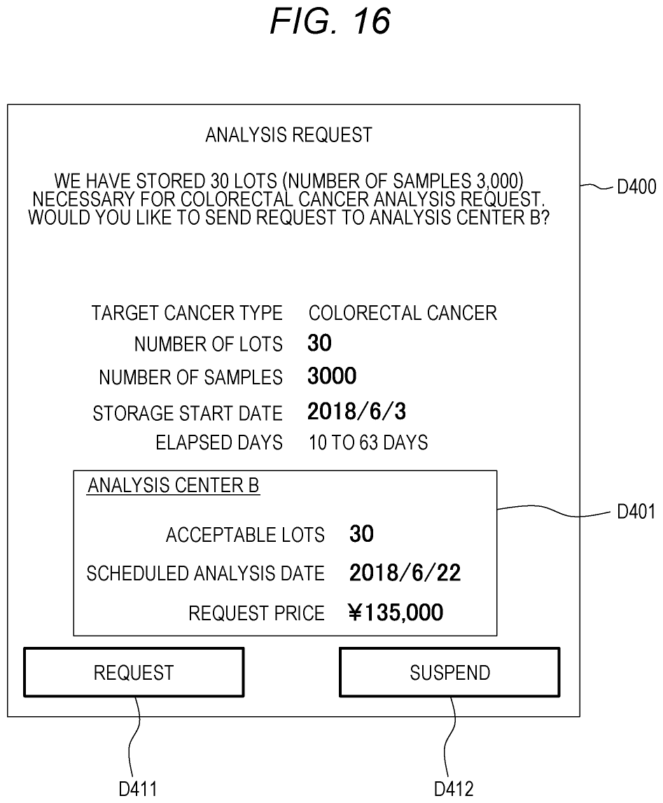

FIG. 16 is a diagram illustrating an example of the analysis request inquiry screen D400.

As illustrated in FIG. 16, information, such as “target cancer type”, “number of lots”, “number of samples”, “storage start date”, and “elapsed days”, is displayed on the analysis request inquiry screen D400.

“Target cancer type” represents a cancer type for inquiring into an analysis request. In the example of FIG. 16, an inquiry is made about a test for “colorectal cancer” is shown.

“Number of lots” represents the number of lots of samples stored for a cancer type for which an inquiry is made about an analysis request. The example illustrated in FIG. 16 represents that 30 lots of samples which are to be tested for “colorectal cancer” are stored.

“Number of samples” represents the number of samples stored for the cancer type for which an analysis request is inquired. The example illustrated in FIG. 16 indicates that 3,000 samples which are to be tested for “colorectal cancer” are stored. Incidentally, as illustrated in FIG. 9, “analysis center B” (analysis center C2b) to which an inquiry is to be made is capable of analyzing 100 samples/lot for the test for “colorectal cancer”. Therefore, “number of samples” illustrated in FIG. 16 shows 30 lots×100 samples/lot=3000 samples are displayed. Note that, referring to FIG. 9, here, the 3,000 samples for which analysis is to be requested have lots (samples) corresponding to the amount of analysis for three days in the “analysis center B” (analysis center C2b). That is, here, the comprehensive analysis center server 1 receives availability information representing that three days are available, from the “analysis center B” (analysis center C2b).

“Storage start date” represents a scheduled date on which storage in the analysis center C2 is started.

“Elapsed days” represents information indicating how many days have elapsed since the sample was collected at the analysis center C2. The example of FIG. 16 shows that some samples having been stored for 10 to 63 days are present in samples for which analysis is to be requested. A staff can select a sample for which no analysis is requested, referring to the number of elapsed days.

In addition, an analysis center information display area D401 displays information (“acceptable lots”, “scheduled analysis date”, “request price”) about an analysis center C2 (“analysis center B” in the example of FIG. 16) as a candidate for performing an analysis. “Acceptable lots” is information displayed referring to “maximum number of lots to be analyzed per day” in the analysis center information database 134 of FIG. 9. “Scheduled analysis date” is information displayed on the basis of the availability received in step S153 of FIG. 3A, and “request price” is information displayed on the basis of the estimate received in step S154.

On the basis of the information displayed on the analysis request inquiry screen D400 of FIG. 16, the staff of the comprehensive analysis center C1 determines whether to make an analysis request or to suspend the analysis request.

When suspending the analysis request, the staff selects a suspend button D412 of FIG. 16 to make an input.

When making an analysis request, the staff selects and the request button D411 of FIG. 16 to make an input.

The description returns to FIG. 3B.

When the staff selects the request button D411 of FIG. 16 to make an input, the comprehensive analysis center server 1 generates lot IDs (S171).

Then, the comprehensive analysis center C1 sends a plurality of frozen samples with sheets having a description of information about test contents, such as the lot IDs and the cancer types tested for, to the analysis center C2 by mail or the like (S172). The plurality of frozen samples sent is referred to as a lot. Note that the information about test contents is preferably described on a sheet in the form of a bar code or the like and attached to a sample container.

At this time, the comprehensive analysis center server 1 inputs data to the columns of “analysis center” and “lot ID” in the sample information database 132 of FIG. 6 with the sample number as a key.

The analysis center C2 receiving the lots of the frozen samples inputs, for example, the test contents read from the attached barcode to the sample information database 132a or 132b illustrated in FIG. 7A or FIG. 7B (S173). At this time, information to be input is “sample acceptance date” and “analysis condition” and the information is input with the sample number or the like stored in the barcode as a key.

Then, in the analysis center C2, setup of a liquid chromatograph mass spectrometer (LC/MS) is started (S181).

Thereafter, liquid chromatography mass spectrometry analysis is performed by the liquid chromatograph mass spectrometer (S182). During the setup, the separation conditions and the measurement conditions described above are set.

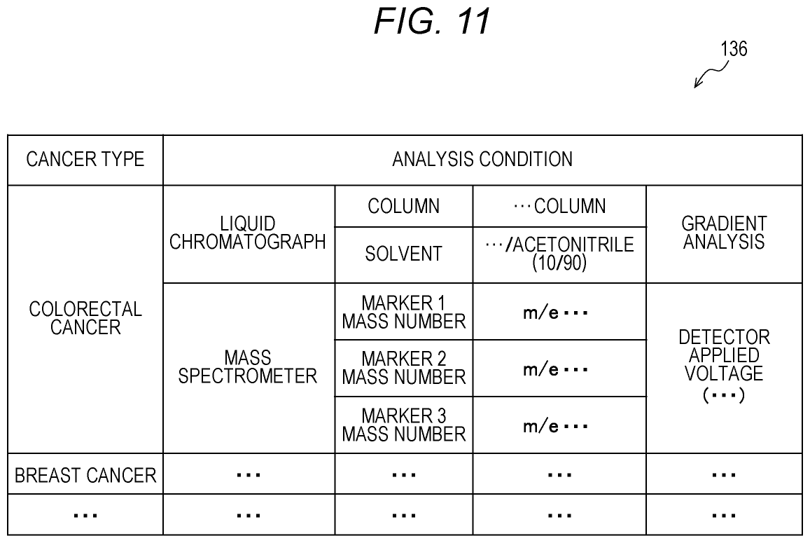

The liquid chromatography mass spectrometry analysis is performed referring to an analysis condition database 136 illustrated in FIG. 11.

(Analysis Condition Database 136)

FIG. 11 is a table illustrating an example of the analysis condition database 136.

The analysis condition database 136 is stored in each analysis center server 2.

As illustrated in FIG. 11, the analysis condition database 136 stores the analysis conditions of the liquid chromatograph and the analysis conditions of mass spectrometer, for each cancer type.

The description returns to FIG. 3B.

Then, the analysis center server 2 acquires analysis results of the liquid chromatograph mass spectrometer (S183).

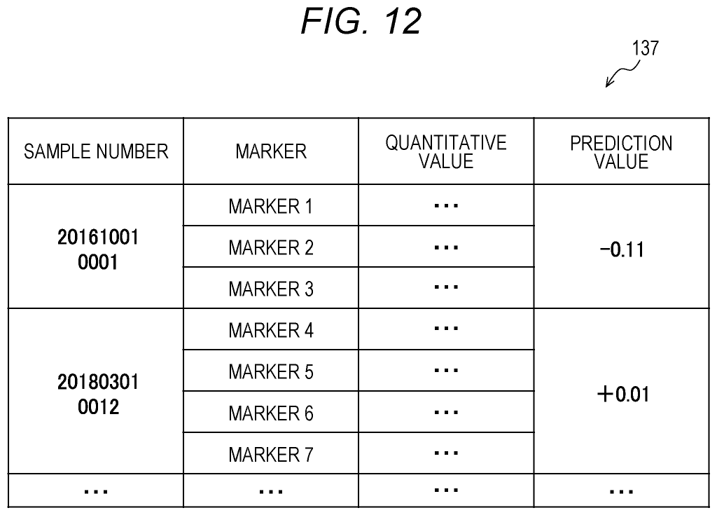

The analysis center server 2 inputs the acquired analysis results to an analysis result information 137 illustrated in FIG. 12 (S184). Note that the analysis of the samples in the analysis center C2 is continuously performed. The analysis at the analysis center C2 will be described later.

(Analysis Result Information 137)

FIG. 12 is a table illustrating an example of the analysis result information 137.

The analysis result information 137 is stored in each analysis center server 2.

As illustrated in FIG. 12, the analysis result information 137 has the column of “sample number”, “marker”, “quantitative value”, and “prediction value”.

“Quantitative value” is a value obtained by the liquid chromatograph mass spectrometer.

“Prediction value” is a value calculated according to the above-described Prediction Formula (1) on the basis of a quantitative value of each marker.

In the present embodiment, each analysis center server 2 calculates a prediction value, but the analysis center server 2 may transmit the quantitative value to the comprehensive analysis center server 1 without calculating the prediction value. Then, the comprehensive analysis center server 1 may calculate the prediction value.

Furthermore, each analysis center server 2 inputs data to “analysis date” and “prediction value” of the sample information database 132a or 132b illustrated in FIG. 7A or FIG. 7B, with the sample number as a key. As the analysis condition, a cancer type tested for is input.

The description returns to FIG. 3B. The analysis center server 2 transmits the analysis result information 137 to the comprehensive analysis center server 1 (S185). The analysis result information 137 to be transmitted may be all of the analysis result information 137 illustrated in FIG. 12 or may be only the prediction value associated with the sample number in FIG. 12. At this time, the analysis center server 2 inputs data to “result transmission date” of the sample information database 132 illustrated in FIG. 6 with the sample number as a key.

The comprehensive analysis center server 1 having acquired the analysis result information 137 performs risk assessment for a target cancer (S191). The risk assessment is performed by comparing a prediction value, which is a test result from the analysis, with a threshold value. The threshold value is determined by a statistical method based on a prediction value in the past and an actual cancer onset status (data group of prediction values of healthy people and cancer patients).

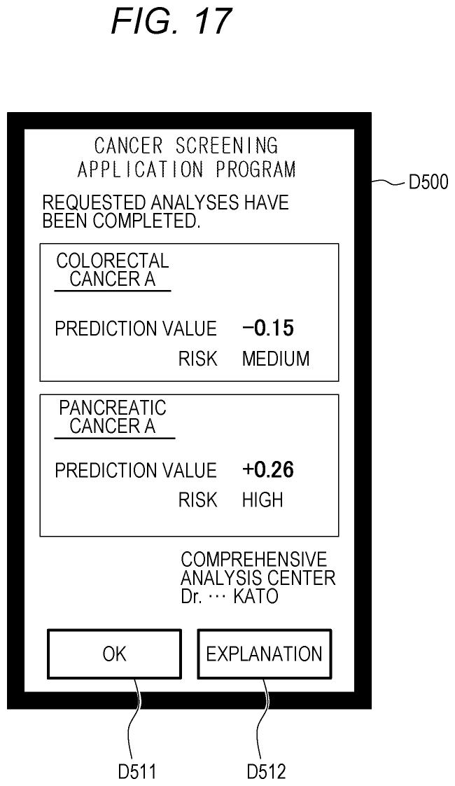

The subject receives a report which says, for example, “you have a “high”, “medium” or “low” risk of developing cancer“, but the wording is not limited to this. For example, in FIG. 17 described later, “risk medium”, “risk high”, and the like are reported.

The threshold value may be determined again by collecting a large amount of data on the prediction values and the cancer onset statuses. Due to the configuration as described above, the accuracy of risk assessment is improved. Determination in step S191 may be performed by each analysis center server 2.

Then, the comprehensive analysis center server 1 inputs the result of step S191 into “risk assessment result” in the sample information database 132 of FIG. 6 with the sample number as a key (S192).

Next, the comprehensive analysis center server 1 confirms whether the tests have been completed for all cancer types for which the subject E has applied (S193). For example, when a subject E desires tests for a plurality of cancer types, such as colorectal cancer and pancreatic cancer, the comprehensive analysis center server 1 confirms whether the tests for all of the cancer types have completed.

After the risk assessment for the cancer, the comprehensive analysis center server 1 transmits a test result to the mobile terminal T of the subject E (S195). At this time, the comprehensive analysis center server 1 transmits the test result to the subject E, referring to the sample information database 132 of FIG. 6 and the membership information database 131 of FIG. 5. Such configuration enables a subject to confirm a test result with his/her mobile terminal T without going to a hospital or the like, and a burden on the subject is reduced.

The application program of the mobile terminal T of the subject E who received the test result displays a test result display screen D500 (see FIG. 17) on the display device (not illustrated) (S196).

Thereafter, the subject E takes action depending on the test result.

(Test Result Display Screen D500)

FIG. 17 is a diagram illustrating an example of the screening result display screen D500.

As illustrated in FIG. 17, on the test result display screen D500, a screening result is displayed, for example, for each cancer type.

When the subject E desires to know the details of the screening result, the user selects an explanation button D512 to make an input. Then, for example, the comprehensive analysis center server 1 transmits information, such as a quantitative value of each marker and a prediction value, to the mobile terminal T. The mobile terminal T displays the information, such as the quantitative value and the prediction value, on the display device (not illustrated) of the mobile terminal T, and simultaneously displays the meanings of such information on the display device.

When the subject E accepts the displayed screening result, the subject E selects an OK button D511 to make an input. Then, information about the selection of the OK button D511 is transmitted to the comprehensive analysis center server 1, and the comprehensive analysis center server 1 determines that the examination of the corresponding sample is completed.

Incidentally, in the example of FIG. 17, the risk of “colorectal cancer” is “medium” and the risk of “pancreatic cancer” is “high”.

In addition, during steps S141 to S195 in FIGS. 3A and 3B, the mobile terminal T of the subject E may display the progress of the screening (S201). For example, information on inquiry about a progress situation is transmitted from the mobile terminal T together with the sample number to the comprehensive analysis center server 1. Then, the comprehensive analysis center server 1 acquires the progress situation of the corresponding sample number and returns information on the acquired progress situation to the mobile terminal T.

(Storage and Management in Comprehensive Analysis Center Server 1)

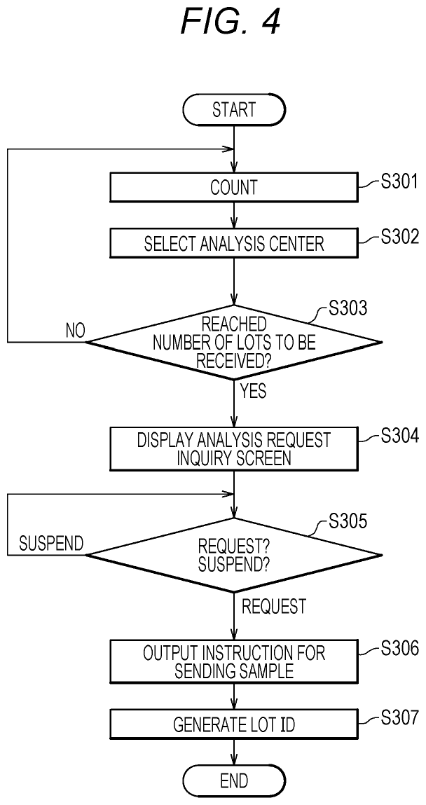

FIG. 4 is a flowchart illustrating a detailed operation procedure of storage and management in the comprehensive analysis center server 1. FIG. 4 illustrates a series of process corresponding to step S143 of FIG. 3A and steps S161 to S171 of FIG. 3B.

Firstly, the counting unit 112 (FIG. 2) counts samples sent, for each cancer type (S301). Here, the counting unit 112 refers to “desired test item” in the membership information database 131 (FIG. 5) with a sample number as a key, identifies a cancer type for which a member corresponding to the sample number desires a test, and then increments the count of the identified cancer type by +1. This process corresponds to the process of step S143 of FIG. 3A.

Next, the center selection unit 113 selects an analysis center C2 on the basis of the availability and the estimate received in step S153 (S302). This process corresponds to the process of step S161 of FIG. 3B. Here, the center selection unit 113 selects an analysis center C2 on the basis of two conditions of (A1) the latest availability and (A2) an inexpensive estimate, for each cancer type. A priority between the conditions (A1) and (A2) is set by a staff (user) of the comprehensive analysis center server 1. The center selection unit 113 selects an analysis center C2 on the basis of the availability and the like of the analysis center C2, and thereby a waiting time in the analysis center C2 is reduced.

Next, the counting unit 112 determines whether the number of lots (the number of samples) for each cancer type in the selected analysis center C2 has reached the number of lots to be received in the analysis center C2 (S303). Step S303 is performed for each of selected analysis centers C2 and cancer types.

As a result of step S303, when the number of lots does not reach the number of lots to be received (S303 No), the counting unit 112 returns the process to step S301.

As a result of step S303, when the number of lots reaches the number of lots to be received (S303 Yes), the input/output processing unit 114 displays the analysis request inquiry screen D400 illustrated in FIG. 16 on the display device 150 (S304). The processing of step S304 corresponds to the processing of step S162 of FIG. 3B.

Then, the input/output processing unit 114 (FIG. 2) determines whether the request button D411 (FIG. 16) of the analysis request inquiry screen D400 is selected to make an input (request?) or whether the suspend button D412 is selected to make an input (suspend?) (S305 in FIG. 4).

As a result of step S305, when the request button D411 is selected to make an input (S305 request), the input/output processing unit 114 (FIG. 2) displays an instruction for sending a sample on the display device 150 (S306). Furthermore, the lot management unit 115 generates lot IDs for a plurality of samples to be sent to the analysis center C2 (S307 in FIG. 4).

A staff sends the samples to the analysis center C2 in accordance with the displayed instruction for sending.

When the suspend button D412 is selected to make an input as a result of step S305 (S305 suspend), the processing unit 111 (FIG. 2) returns the process to step S305.

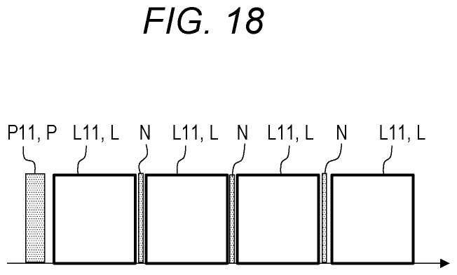

Next, the effects of the cancer screening system Z according to the present embodiment will be described with reference to FIG. 18.

FIG. 18 illustrates an example of testing lots for the same cancer type.

As illustrated in FIG. 18, when lot processing L11 (L) is continuously performed for the same cancer type (for example, “cancer type A”), only the first adjustment P (adjustment P11) is required. Thus, compared to the method illustrated in FIG. 24, the time required for the adjustment P is significantly reduced. Furthermore, as illustrated in FIG. 18, the number of lots is determined in accordance with the condition check N, and a time between the lot processing L and the condition check N is significantly reduced compared to the example illustrated in FIG. 24.

Such processing increases the operation rate of the liquid chromatograph mass spectrometer to approximately 90 percent per week.

That is, in the present embodiment, the comprehensive analysis center C1 stores samples up to a predetermined number, and thereafter, the samples are sent to the analysis center C2 to be continuously analyzed for tests for the same cancer type. Such a configuration greatly improves analysis efficiency as illustrated in FIG. 18.

### Second Embodiment

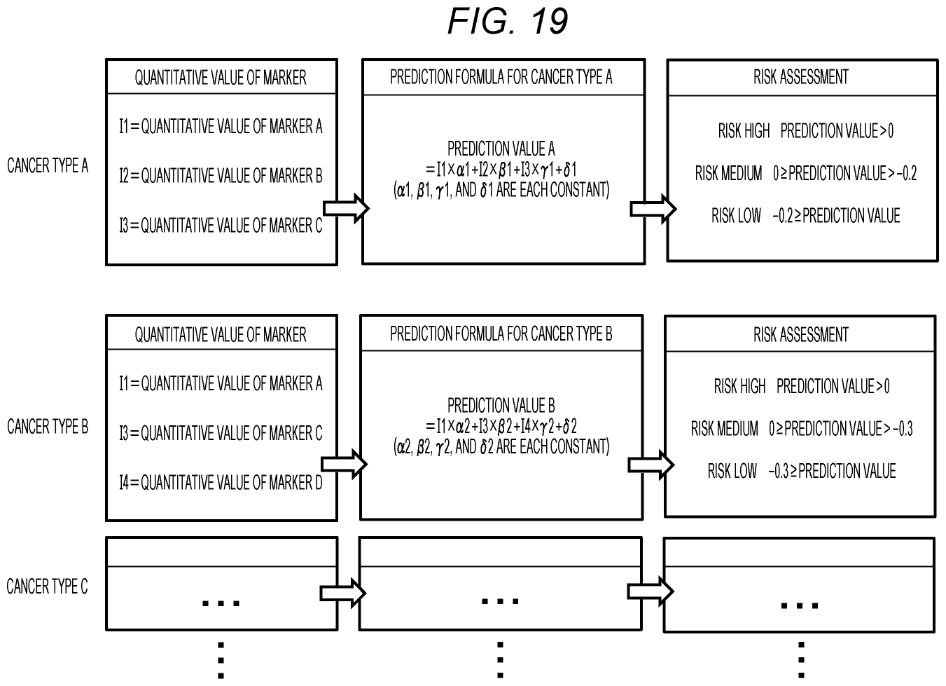

Next, a second embodiment of the present invention will be described with reference to FIGS. 19 to 22.

FIG. 19 is a diagram illustrating a test method for different cancer types.

In a cancer screening using a marker (urinary tumor marker), it is possible to change the cancer type tested for may be changed by changing the type of marker used. For example, as illustrated in FIG. 19, when three types of markers are used for “cancer type A,” quantitative values I1 to 13 of markers A to C obtained as a result of analysis are used to calculate “prediction value A” according to a prediction formula for the cancer type A illustrated in FIG. 19. Then, the risk assessment of “cancer type A” is performed on the basis of this “prediction value A”. In the example of FIG. 19, when prediction value A>0, it is determined that the risk of cancer (“cancer type A”) is high. Likewise, when 0 prediction value A>−0.2, it is determined that the risk of cancer is “medium”, and when −0.2 prediction value A, the risk of cancer is low. Here, “0, −0.2” are referred to as threshold values for the cancer type A.

Likewise, for “cancer type B”, “prediction value B” is calculated according to a prediction formula for the cancer type B illustrated in FIG. 19, on the basis of quantitative values I1, I3, and I4 of the markers A and C and a marker D. Then, the risk assessment of “cancer type B” is performed on the basis of this “prediction value B”. In the example of FIG. 19, when the prediction value B>0, it is determined that the risk of cancer (“cancer type B”) is high. Likewise, when 0≥prediction value B>−0.3, it is determined that the risk of cancer is “medium”, and when −0.3≥prediction value B, the risk of cancer is low. Here, “0, −0.3” are referred to as threshold values for the cancer type B.

As described above, threshold values are determined by a statistical method based on a prediction value in the past and an actual cancer onset status (data group of prediction values of healthy people and cancer patients).

Likewise, also for “cancer type C”, “prediction value C” is calculated according to a prediction formula for the “cancer type C”. Then, the risk assessment of “cancer type C” is performed on the basis of a threshold value set to “prediction value C”. The risk assessment is also performed in the same manner for “cancer type D”, “cancer type E”, which are not illustrated.

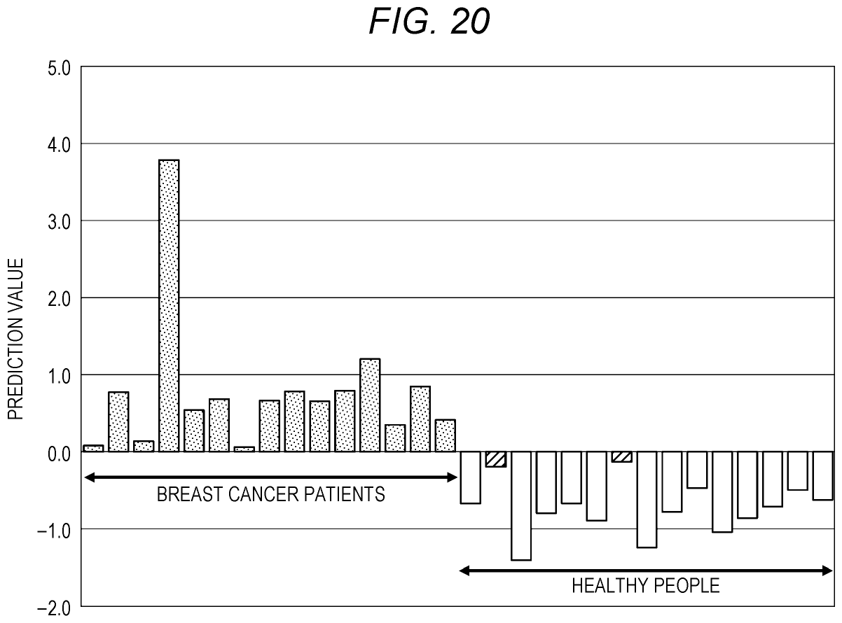

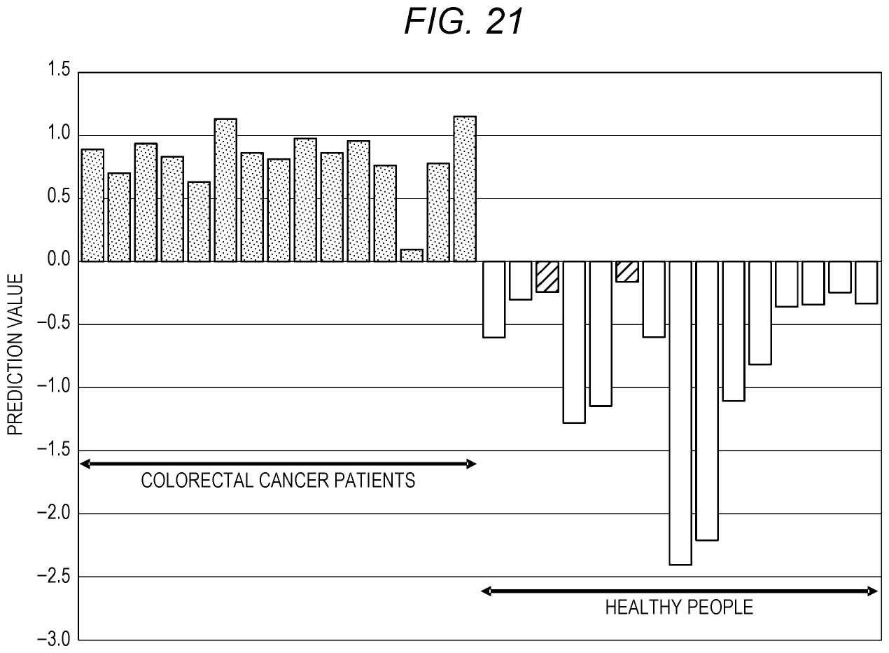

FIG. 20 is a graph illustrating a result of risk assessment for breast cancer performed by a method as illustrated in FIG. 19. FIG. 21 is a graph illustrating a result of risk assessment for colorectal cancer performed by the method as illustrated in FIG. 19. That is, for breast cancer, for example, risk assessment is performed by using the method for “cancer type A” of FIG. 19, and for colorectal cancer, for example, risk assessment is performed by the method for “cancer type B” of FIG. 19.

In the bar graphs of FIGS. 20 and 21, “risk high” is indicated by dots, “risk medium” is indicated by oblique lines, and “risk low” is indicated by outline. Incidentally, in FIGS. 20 and 21, “risk medium” is within the range of healthy people.

Moreover, in FIGS. 20 and 21, the risk assessment is performed for obtained prediction values, by using past actual statistical data of urine samples (cancer patients, healthy people).

That is, in FIGS. 20 and 21, prediction values of the respective cancer types are calculated on the basis of actual measurement data, showing the prediction values of breast cancer patients (“stage 2” and “stage 3”) and healthy people and colorectal cancer patients (“stage 2” and “stage 3”) and healthy people.

In such a method, a threshold value of each cancer type is determined by a statistical method using a large number of urine samples (of both of cancer patients and healthy people). Thus, the greater the number of urine samples, the more accurate the risk assessment. In addition, the accuracy of the risk assessment may improve by changing the threshold value as the number of urine samples collected increases.

Heretofore, when a subject E desires tests for a plurality of cancer types, one computer carries out the tests as illustrated in FIG. 19 for a plurality of cancer types. However, as the number of cancer types tested for increases, a program becomes complicated, and thus program errors are likely to occur.

[Cancer Screening System Za]

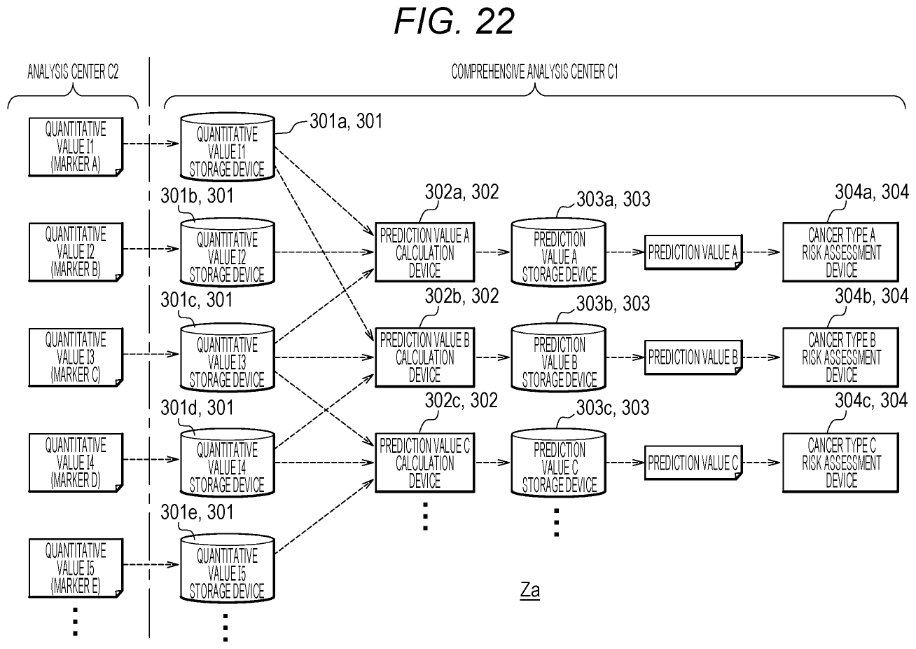

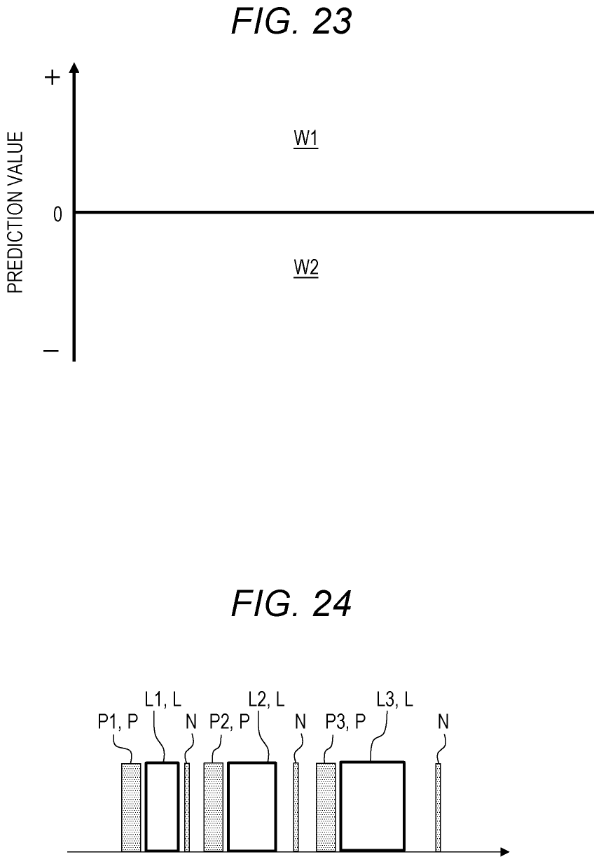

In order to solve such a problem, a cancer screening system Za illustrated in FIG. 22 will be presented.

Unlike the cancer screening system Z described in the first embodiment, in the cancer screening system Za, a prediction value is calculated in a comprehensive analysis center C1. Note that, in FIG. 22, illustration of a mobile terminal T is omitted.

The comprehensive analysis center C1 includes quantitative value storage devices 301, prediction value calculation devices 302, prediction value storage devices 303, and risk assessment devices 304. In addition, the comprehensive analysis center C1 includes a comprehensive analysis center server 1 (see FIG. 1) provided separately from these devices, which is not illustrated in FIG. 22.

Firstly, in an analysis center C2, urine samples of the same subject E are analyzed for respective types of markers, and a quantitative value of each type of marker is transmitted to the comprehensive analysis center C1 as the result of the analysis. This processing corresponds to the processing of step S185 of FIG. 3B. However here, only the quantitative value is transmitted although the prediction value is transmitted in step S185 of FIG. 3B.

The transmitted quantitative values are stored in quantitative value storage devices 301 of the comprehensive analysis center C1.

The quantitative value storage devices 301 include a quantitative-value-I1 storage device 301a, a quantitative-value-I2 storage device 301b, each of which is an independent storage device for storing a quantitative value corresponding to each marker and transmitted from the analysis center C2. That is, the quantitative-value-I1 storage device 301a stores quantitative values I1 obtained from various urine samples. The same applies to the quantitative-value-I2 storage device 301b and following storage devices.

Then, each prediction value calculation device 302 calculates a prediction value on the basis of the quantitative values obtained from the same subject E and stored in the quantitative value storage devices 301. As illustrated in FIG. 22, the prediction value calculation devices 302 include a prediction-value-A calculation device 302a, a prediction-value-B calculation device 302b, a prediction-value-C calculation device 302c and the like.

Then each prediction value calculation device 302 combines quantitative values on the basis of a cancer type for which the prediction value is to be calculated, for the same subject E, and a prediction value corresponding to each cancer type is calculated.

For example, when calculating “prediction value A” corresponding to “cancer type A” described above, the prediction-value-A calculation device 302a calculates “prediction value A” according to the prediction formula for the cancer type A (see FIG. 19) using quantitative values I1 to I3. The quantitative values I1 to I3 are acquired from the quantitative-value-I1 storage device 301a, the quantitative-value-I2 storage device 301b, and the quantitative-value-I3 storage device 301c.

In addition, when calculating “prediction value B” corresponding to “cancer type B” described above, the prediction-value-B calculation device 302b calculates “prediction value B” according to the prediction formula for the cancer type B (see FIG. 19) using quantitative values I1, I3, and I4. The quantitative values I1, I3, and I4 are acquired from the quantitative-value-I1 storage device 301a, the quantitative-value-I3 storage device 301c, and the quantitative-value-I4 storage device 301d.

Then, when calculating “prediction value C” corresponding to “cancer type C, the prediction-value-C calculation device 302c calculates “prediction value C” according to a prediction formula for the cancer type C (not illustrated) using quantitative values I3 to I5. The quantitative values I3 to I5 are acquired from the quantitative-value-I3 storage device 301c, the quantitative-value-I4 storage device 301d, and the quantitative-value-I5 storage device 301e.

Note that a prediction value to be calculated is determined by a staff of the comprehensive analysis center C1 depending on a cancer type tested for which the subject E desires.

In addition, in analyzing various markers, all markers may be analyzed regardless of the desire of the subject E, but analyzing markers relating to a cancer type selected by the subject E is preferable in terms of cost to analyze.

The calculated various prediction values are stored in prediction value storage devices 303. The prediction value storage devices 303 include a prediction-value-A storage device 303a configured to store “prediction value A”, a prediction-value-B storage device 303b configured to store “prediction value B”, and a prediction-value-C storage device 303c configured to store “prediction value C”.

Then, the risk assessment devices 304 perform risk assessment by using various prediction values stored in each prediction value storage device 303 (corresponding to step S191 of FIG. 3B).

Here, as illustrated in FIG. 22, the risk assessment devices 304 include a cancer-type-A risk assessment device 304a, a cancer-type-B risk assessment device 304b, a cancer-type-C risk assessment device 304c and the like.

The cancer-type-A risk assessment device 304a performs risk assessment of “cancer type A” by using “prediction value A” stored in the prediction-value-A storage device 303a and a threshold value for the cancer type A.

The cancer-type-B risk assessment device 304b performs risk assessment of “cancer type B” by using “prediction value B” stored in the prediction-value-B storage device 303b and a threshold value for the cancer type B.

Then, the cancer-type-C risk assessment device 304c performs risk assessment of “cancer type C” by using “prediction value C” stored in the prediction-value-C storage device 303c and a threshold value for the cancer type C.

Applying such analyses to a plurality of subjects E, it is possible to perform risk assessment for a plurality of cancer types, for each of the plurality of subjects E.

As described above, in the present embodiment, a so-called IoT platform is interposed, and dedicated devices, such as the prediction-value-A calculation device 302a, the prediction-value-B calculation device 302b, the prediction-value-C calculation device 302c, the cancer-type-A risk assessment device 304a, the cancer-type-B risk assessment device 304b, and the cancer-type-C risk assessment device 304c, are prepared for each cancer type. Even when an analysis for a new cancer type is developed, such a configuration enables to add another quantitative value storage device 301, prediction value calculation device 302, prediction value storage device 303, and risk assessment device 304 for the new cancer type to readily respond to the analysis for a new cancer type. In addition, program errors can be prevented.

Furthermore, even when it is found that, with study progress, the accuracy of an existing cancer type test is improved by combining a new quantitative value, the cancer test improved in accuracy is readily performed by changing the connection between the quantitative value storage devices 301 and the prediction value calculation devices 302.

As described above, not only when the number of subject E increases but also when types of cancer tested for increase the cancer screening system Za as described in the second embodiment enables facilitate analysis by increasing the number of the quantitative value storage devices 301, the prediction value calculation devices 302, the prediction value storage devices 303, and the risk assessment devices 304.

Note that the present invention is not limited to the above embodiments and may include various modifications. For example, the above embodiments are described in detail for an easy understanding of the present invention and is not necessarily limited to all the configurations described above. Furthermore, part of a configuration of an embodiment may be replaced with a configuration of another embodiment, and a configuration of an embodiment may be added to a configuration of another embodiment. Still furthermore, for part of a configuration of each embodiment, an addition, elimination, or substitution of another configuration may be made.

Furthermore, each of the above-described configurations, functions, units 111 to 115, database 130, devices 131 to 134, and the like may be partially or wholly achieved by hardware, such as a designed integrated circuit. In addition, as illustrated in FIG. 2, each of the above-described configurations, functions, and the like may be achieved by software so that a processor, such as the CPU 120, may interpret and execute a program for achieving each function. Information, such as a program, table, and file, for achieving each function may be stored in a recording device, such as the memory 110 or a solid state drive (SSD), or a recording medium, such as an integrated circuit (IC) card, secure digital (SD) card, or digital versatile disc (DVD), in addition to a hard disk (HD).

In the above embodiments, control lines and information lines considered to be necessary for description are described, and all control lines and information lines required for a product are not always described. In practice, it may be considered that almost all the configurations are mutually connected.

Although the invention has been described above by reference to certain embodiments of the invention, the invention is not limited to the embodiments described above. Modifications and variations of the embodiments described above will occur to those skilled in the art, in light of the above teachings. The scope of the invention is defined with reference to the following claims.

What is claimed is:

1. A cancer screening processor comprising:
a storage unit storing sample information including a piece of information on a number of urine samples sent from subjects,
a center selection unit selecting an analysis center for each of cancer type on which cancer screening is perform on the basis of the cancer type and analysis center information transmitted from analysis centers;
the analysis center
receiving a sample container containing the urine samples being dispensed for a cancer type,
analyzing a urinary tumor marker in the dispensed urine sample for the cancer type, and
a processing unit counting a number of the sample containers and performing a process step of sending the sample containers to the analysis center when the number of sample containers becomes equal to a number in the analysis center information transmitted from the analysis center.

2. The cancer screening processor as claimed in claim 1 comprising:
a display processing unit displaying an image of a urine collection container on a display unit;
the image being sent from a user device of the subject and,
the urine collection container containing urine collected from the subject.

3. The cancer screening processor as claimed in claim 1 comprising:
the center selection unit selecting the analysis center by referring to the analysis center information including availability of analyses at the analysis center, from analysis center.

4. The cancer screening processor as claimed in claim 1, further comprising:
a transmission/reception unit receiving an analysis result from the analysis center and transmitting a result of a risk assessment for the cancer to a user device of the subject;
the result of the risk assessment being determined on the basis of the analysis results.

5. The cancer screening processor as claimed in claim 4, wherein:
the analysis result is a quantitative value of the urinary tumor marker for the cancer,
the cancer screening processor assesses a risk of the cancer by comparing a prediction value needed for the risk assessment for the cancer with a threshold value; the prediction value being calculated on the basis of the quantitative value, and
the threshold value is determined by the prediction values in the past and a group of the prediction values of the healthy people and cancer patients.

6. A cancer screening system comprising:
a cancer screening processor comprising:
a storage unit storing sample information including a piece of information on a number of urine samples sent from subjects,
a center selection unit selecting an analysis center for each of cancer type on which cancer screening is to be perform on the basis of the cancer type and analysis center information transmitted from analysis centers;
the analysis center
receiving a sample container containing the urine samples being dispensed for the cancer type, and
analyzing a urinary tumor marker in the dispensed urine sample for the cancer type,
a processing unit counting a number of the sample containers and performing a process step of sending the sample containers to the analysis center when the number of sample containers becomes equal to a number in the analysis center information transmitted from the analysis center, and,
an analysis center server
being installed in each of the analysis centers where a liquid chromatograph mass spectrometer is installed and the dispensed urine sample is analyzed by the liquid chromatograph mass spectrometer, and,
receiving inquiry information transmitted from the cancer screening processor in the process step of sending the sample containers.

7. The cancer screening system as claimed in claim 6, wherein
an analysis result from the analysis center is a quantitative value of the urinary tumor marker for the cancer type,
the cancer screening processor assesses a risk of the cancer by comparing a prediction value needed for a risk assessment for the cancer with each of a threshold value of the cancer; the prediction value being calculated on the basis of the quantitative value, and
the threshold value is determined by the prediction values in the past and a group of the prediction values of the healthy people and cancer patients.

8. The cancer screening system as claimed in claim 6, wherein
an analysis result in the analysis center is a quantitative value of the urinary tumor marker;
the cancer screening system further comprising:
quantitative value storage devices, each quantitative value storage device corresponding to a quantitative value of a urinary tumor marker for a cancer type
prediction value calculation devices, prediction value storage devices, and risk assessment devices; each of the devices corresponding to a cancer type,
each of the prediction value calculation devices calculates a prediction value necessary for a risk assessment for a cancer type on the basis of a combination of the quantitative values;
the prediction value needed for a risk assessment being calculated for the cancer type corresponding to the prediction value storage device itself,
each of the prediction value storage devices stores the prediction value necessary for a risk assessment for the cancer type corresponding to the prediction value storage device itself, and
each of the risk assessment devices assesses a risk of a cancer by comparing the prediction value for the cancer type corresponding to the risk assessment device itself with a threshold value for the cancer type.

9. The cancer screening system as claimed in claim 8, wherein
the threshold value is determined by the prediction values in the past and a group of the prediction values of the healthy people and cancer patients.

10. A cancer screening processing method comprising,
a cancer screening processor comprising a storage unit storing sample information including a piece of information on a number of urine samples sent from subjects, managing a sample for a cancer screening analyzing with a urinary tumor marker, and performing a process step relating to the cancer screening,
selecting an analysis center for each cancer type on which cancer screening is to be perform on the basis of the cancer type and analysis center information from analysis centers,
the analysis center
receiving a sample container containing the urine sample being dispensed for the cancer type,
analyzing the urinary tumor marker in the dispensed urine sample for the cancer type,
counting a number of the sample containers on the basis of the sample information, and
performing a process step of sending the sample container to the analysis center when the number of the sample containers becomes equal to a number in the analysis center information transmitted from the analysis center.
